(19) United States
(12) Patent Application Publication
(10) Pub. No.: US 2004/0236146 A1
(43) Pub. Date: Nov. 25, 2004

(54) Method for producing 3-bromomethylbenzoic acids

(76) Inventor: Hansjorg Lehmann, Wutoschingen (DE)

Correspondence Address: CONNOLLY BOVE LODGE & HUTZ, LLP, P O BOX 2207, WILMINGTON, DE, 19899, US

(21) Appl. No.: 10/489,142

(22) PCT Filed: Aug. 29, 2002
(86) PCT No.: PCT/EP02/09630

(30) Foreign Application Priority Data
Sep. 11, 2001 (DE) 101444125

(51) Int. Cl. C07C 315/04

(52) U.S. Cl. 562/429

(57) ABSTRACT

A process is described for preparing 3-bromomethylbenzoic acids of the formula II

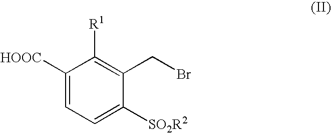

by brominating the corresponding 3-methylbenzoic acids.

In addition, compounds of the formula II are described where R₁ is fluorine chlorine or bromine, and R₂ is (C₁-C₄)alkyl.

The invention relates to a process for preparing 3-bromomethylbenzoic acids by brominating the corresponding 3-methylbenzoic acids. It further relates to certain 3-bromomethylbenzoic acids.

Derivatives of 3-bromomethylbenzoic acid constitute valuable reactants in the synthesis of certain herbicides. Bromomethyl aromatics may in principle be prepared from the corresponding methyl aromatics by side chain bromination; on this subject, cf. Houben-Weyl, Vol. 5, page 331 ff. (1960). However, it is known that electronegative substituents such as carboxyl, alkylcarbonyl, cyano and nitro distinctly hinder the reaction, so that only poor yields can be achieved. WO 99/06339 discloses a process for preparing substituted benzyl bromides by brominating the corresponding methyl aromatics in the presence of azocarboxylic esters or azonitriles and in the presence of an oxidizing agent. In this case, one substituent is electronegative and is from the group fluorine, chlorine, bromine, alkoxycarbonyl, cyano and nitro. Disadvantages of this process are the use of an additional oxidizing agent and the not always satisfactory yields. EP-A 0 292 944 describes the preparation of methyl 3-bromomethyl-2-chloro-4-methylsulfonylbenzoate by radical initiator-induced bromination of methyl 2-chloro-3-methyl-4-methylsulfonylbenzoate in carbon tetrachloride. The free acid, 3-bromomethyl-2-chloro4-methylsulfonylbenzoic acid is then obtained from this latter compound by hydrolysis.

Specifically with regard to the preparation of certain herbicides, processes for directly preparing 3-bromomethylbenzoic acids are desirable. It has now been found that 2-halo-3-methyl4-alkylsulfonylbenzoic acids can be converted in very good yields and in very high purities by bromination to the corresponding 3-bromomethyl-2-halo-4-alkylsulfonylbenzoic acids.

The present invention therefore provides a process for preparing 3-bromomethylbenzoic acids of the formula II

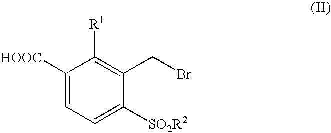

by brominating 3-methylbenzoic acids of the formula I

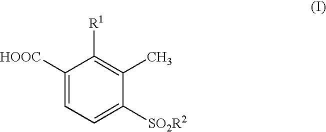

with

A) N-bromosuccimide in the presence of a radical initiator, or

B) elemental bromine and irradiation with a photolamp,

where, in formulae I and II,

R¹ is fluorine, chlorine or bromine, and

R² is (C₁-C₄)alkyl.

Suitable radical initiators for the bromination of variant A) are radical initiators which are commercially available and known to those skilled in the art, such as diaroyl peroxides, azocarboxylic esters and azonitriles. Examples include azoisobutyronitrile and dibenzoyl peroxide. Radiation with light or variant B) is effected with commercially available photolamps and is known in principle to those skilled in the art.

In addition to the very high yields and very high purities, further advantages of the process according to the invention may be regarded as being the use of less toxic solvents and the particularly simple workup of the end products.

Suitable solvents for the process according to the invention have been found to be those which can be regarded as being inert with respect to the conditions of the bromination. These include, for example, halogenated hydrocarbons such as methylene chloride, chloroform, carbon tetrachloride, 1,2-dichloroethane and chlorobenzene, and also compounds such as acetonitrile. It has been found that the solvents chlorobenzene and acetonitrile are advantageous for process variant A), and the solvents chlorobenzene, methylene chloride and 1,2-dichloroethane for process variant B). It will be appreciated that mixtures of these solvents may also be used.

In both process variants, the reaction is generally carried out at a temperature of from 40 to 100° C., preferably from 70 to 100° C. In the case of the low-boiling solvents acetonitrile, 1,2-dichloroethane and methylene chloride, preference is given to working under reflux.

In process variant A), it is appropriate to initially charge a compound of the formula I with N-bromosuccinimide (NBS) and radical initiator in solvent, and then to heat gradually. The reaction may optionally be accelerated by adding a little bromine. Preference is given to using NBS in excess.

In process variant B), it is appropriate to initially charge a compound of the formula I in solvent and then, after heating under irradiation with a photolamp, to add bromine dropwise. Preference is given to using bromine in excess.

Depending on the solubilities of the compounds of the formulae I and II in the solvent used, these compounds are completely or partly dissolved. In general, they are partly dissolved, so that a portion is in suspended. Typically, the reaction in both process variants has proceeded to completion after from about 2 to 6 hours. The precise time at which the reaction is completed may be monitored, for example, by thin-film chromatography or by HPLC.

For workup, the reaction mixture is allowed to cool. When the bromination is effected by process variant A), it is appropriate to initially add sodium bisulfate solution and optionally solvents. The further workup then depends upon the solubility and purity of the product. In the case of process variant A), the reaction product may generally be directly filtered off with suction after cooling. In both cases, the reaction product is washed and, if necessary, further purified by recrystallization, for example from ethyl acetate or butyl acetate.

With the exception of the compound 3-bromomethyl-2-chloro4-methylsulfonylbenzoic acid which is disclosed by EP-A 0 292 944, compounds of the formula II are novel. The present invention therefore further provides compounds of the formula II

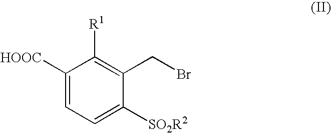

where

R¹ is fluorine, chlorine or bromine, and

R² is (C₁-C₄)alkyl,

excluding the compound 3-bromomethyl-2-chloro4-methylsulfonylbenzoic acid.

The inventive examples which follow illustrate the invention:

### EXAMPLE 1

### Preparation of 3-bromomethyl-2-chloro-4-methylsulfonylbenzoic Acid

Process Variant A):

In a 1 I stirred apparatus, 100g of 2-chloro-3-methyl4-sulfonylmethylbenzoic acid were suspended in 400 ml of chlorobenzene. At room temperature, 85.9 g of N-bromosuccinimide and 6.6 g of azoisobutyroniltrile were added and the mixture was subsequently heated to 60° C. Afterwards, 2.6 g of bromine were added and the reaction mixture was further heated gradually to 90° C. The mixture was stirred at 90° C. for 2 h; after this time, HPLC analysis indicated a conversion of more than 98%. After the reaction mixture had been cooled, 100 ml of a 2% sodium bisulfite solution and 400 ml of water were added in succession and stirred well. The suspension was filtered, and the solid was washed and dried at 60° C. under reduced pressure.

121.3 g (88.4% of theory) of 3-bromomethyl-2-chloro4-methylsulfonyl-benzoic acid were obtained.

Purity: 96% (HPLC) Melting point: 207-208° C.

Process Variant B):

A 250 ml glass flask was initially charged with 5 g of 2-chloro-3-methyl-4-sulfonylmethylbenzoic acid in 50 ml of methylene chloride and 50 ml of water. Under irradiation with a 300 W lamp, II g of bromine were gradually metered in over 3 h, and the mixture boiled under reflux. After a further 2 h of irradiation, the reaction mixture had been substantially decolorized. According to HPLC analysis, the reaction mixture contained 87% of product and 10% of reactant.

### EXAMPLE 2

Process Variant A):

### Preparation of 3-bromomethyl-2-chloro-4-ethylsulfonylbenzoic Acid

In a 0.5 I stirred apparatus, 20 g of 2-chloro-3-methyl-4-sulfonylethylbenzoic acid were suspended in 150 ml of acetonitrile. At room temperature, 17.6 g of N-bromosuccinimide and 0.7 g of dibenzoyl peroxide were added and the mixture was subsequently heated to reflux. After in each case 1 h, further dibenzoyl peroxide was added in 3 portions each of 0.7 g and the mixture was stirred for a further 2 h after the last addition. After this time, HPLC analysis indicated complete conversion. After the reaction mixture had been cooled, 100 ml of a 2% sodium bisulfite solution and 300 ml of ethyl acetate were added in succession. The organic phase was removed, washed and concentrated under reduced pressure. The solid residue was suspended in 50 ml of diethyl ether and 20 ml of heptane, filtered off and dried. 28.2 g (95.6% of theory) of 3-bromomethyl-2-chloro-4-ethylsulfonylbenzoic acid were obtained.

Purity: 88% (HPLC) Melting point: 103° C.

1-5 (Canceled)

6. A process for preparing a 3-bromomethylbenzoic acid of formula II 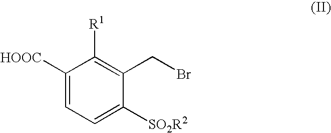
which comprises brominating a 3-methylbenzoic acid of formula I 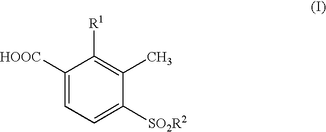
with N-bromosuccimide in the presence of a radical initiator,
where, in formulae I and II
R¹ is fluorine, chlorine or bromine, and
R² is (C₁-C₄)alkyl.

7. The process as claimed in claim 6, wherein the radical initiator is azoisobutyronitrile or dibenzoyl peroxide.

8. The process as claimed in claim 6, wherein a solvent is used.

9. The process as claimed in claim 8, wherein the solvent is chlorobenzene.

10. The process as claimed in claim 8, wherein the solvent is acetonitrile.

11. The process as claimed in claim 6, wherein the brominating is carried out at a temperature of from 70 to 100° C.

12. The process as claimed in claim 10, wherein the brominating reaction is carried out at the reflux temperature of the solvent.

13. A process for preparing a 3-bromomethylbenzoic acid of formula II 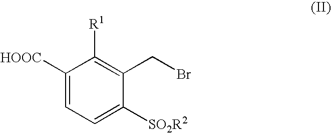
which comprises brominating a 3-methylbenzoic acid of formula I 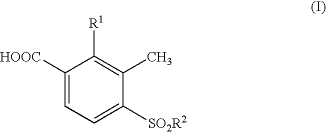
with elemental bromine and irradiation with a photolamp,
where, in formulae I and II
R¹ is fluorine, chlorine or bromine, and
R² is (C₁-C₄)alkyl.

14. The process as claimed in claim 13, wherein a solvent is used.

15. The process as claimed in claim 14, wherein the solvent is chlorobenzene.

16. The process as claimed in claim 14, wherein the solvent is methylene chloride.

17. The process as claimed in claim 13, wherein the brominating is carried out at a temperature of from 70 to 100° C.

18. The process as claimed in claim 16, wherein the brominating is carried out at the reflux temperature of the solvent.

19. A compound of formula II 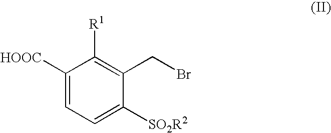
wherein
R¹ is fluorine, chlorine or bromine, and
R² is (C₁-C₄)alkyl,
excluding 3-bromomethyl-2-chloro-4-methylsulfonylbenzoic acid.

20. The compound as claimed in claim 19, wherein
R¹ is chlorine, and
R² is (C₂)alkyl.
